(19) United States
(12) Patent Application Publication
(10) Pub. No.: US 2023/0200652 A1
(43) Pub. Date: Jun. 29, 2023

(54) SYSTEM TO DETECT FOOT ABNORMALITIES

(71) Applicant: EMPO HEALTH, INC., San Bruno, CA (US)

(72) Inventors: Anuj KHANDELWAL, Palo Alto, CA (US); Eric DAHLSENG, San Francisco, CA (US)

(21) Appl. No.: 18/172,207

(22) Filed: Feb. 21, 2023

Related U.S. Application Data
(63) Continuation of application No. PCT/US2021/046978, filed on Aug. 20, 2021.
(60) Provisional application No. 63/068,567, filed on Aug. 21, 2020.

(51) Int. Cl. A61B 5/00

(52) U.S. Cl. CPC A61B 5/004; A61B 5/6829; A61B 5/746

(57) ABSTRACT

A device for detecting a foot abnormality includes a platform configured to be stood upon by a user, an imaging device within the platform, and a processor connected to the imaging device. The processor is configured to detect a foot abnormality from images gathered by the imaging device.

### CROSS REFERENCE TO RELATED APPLICATIONS

This application claims priority as a continuation of PCT Application No. PCT/US2021/046978, filed Aug. 20, 2021, which application claims priority to U.S. Provisional Patent Application No. 63/068,567 filed Aug. 21, 2020, the entirety of each is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Many types of foot complications, particularly when left untreated, can lead to serious issues in patients that progress through the various layers of tissue in the foot, even affecting bones. Foot complications that progress too far ultimately lead to amputations. Early intervention by medical professionals is often critical for ensuring that foot complications heal properly.

While foot complications can be caused by a number of different factors, they are often associated with diabetes and diabetic neuropathy. Patients with diabetic neuropathy usually have decreased sensation in their feet. This decreased sensation makes it difficult for these patients to feel foot complications as they develop, allowing foot complications to easily go unnoticed in the early stages.

Doctors typically examine the feet of at-risk patients for foot complications during routine visits. However, given the relatively low frequency of doctor visits compared with the rate of progression of many foot complications, doctors often must rely on self-examinations by patients at home in order to catch foot complications as they develop. Unfortunately, many patients are unable to view all parts of their feet, don't understand what they are looking for, or simply forget to do their self-examinations.

Accordingly, a system or device to examine patients' feet for complications (outside of a doctor's office, such as in a patient's home) in a reliable and frequent way is desired so as to promote key medical interventions more quickly than relying on patient self-examinations alone.

### SUMMARY OF THE DISCLOSURE

In general, in one embodiment, a device for detecting a foot abnormality (e.g., complication) includes a platform configured to be stood upon by a user, an imaging device within the platform, and a processor connected to the imaging device. The imaging device includes a large area imaging sensor configured to image a foot of a user standing on the platform. The processor is configured to detect a foot complication from images gathered by the imaging device.

In general, in one embodiment, a device for detecting a foot abnormality (e.g., complication) includes a bathmat configured to be stood upon by a user, an imaging device within the bathmat, and a processor connected to the imaging device. The imaging device is configured to image a foot of a user standing on the platform. The processor is configured to detect a foot abnormality (e.g., complication) from images gathered by the imaging device.

In general, in one embodiment, a system for detecting a foot abnormality includes a platform configured for engagement with a foot of a user, an imaging device within the platform having a large area imaging sensor configured to image the foot of the user while the foot is engaged with the platform, and a connector connected to the imaging device configured to communicate with a processor to detect a foot abnormality from a plurality of images gathered by the imaging device.

In general, in one embodiment, a system for detecting a foot abnormality includes a bathmat platform configured for engagement with a foot of a user, an imaging device within the bathmat platform configured to image the foot of the user while the foot is engaged with the bathmat platform, and a connector connected to the imaging device configured to communicate with a processor to detect a foot abnormality from a plurality of images gathered by the imaging device.

In general, in one embodiment, a system for detecting a foot abnormality includes a platform configured for engagement with a foot of a user, an imaging device within the platform, a sensor in or on the platform and configured to detect that the foot of the user has engaged with the platform, and a connector connected to the imaging device and the sensor, wherein the connector is configured to communicate with a processor to detect a foot abnormality from a plurality of images gathered by the imaging device. The imaging device is configured to automatically image the foot of the user after the sensor has detected that the foot of the user has engaged with the platform

This and other embodiments can include one or more of the following features. The imaging device can include a large area imaging sensor configured to image the foot of the user while the foot is engaged with the platform. The system can further include the processor. The processor can be in the platform. The processor can be remote from the platform. The processor can be configured to issue an alert flag indicating suspicion of a foot abnormality based on the plurality of images gathered by the imaging device. The system can further include a plurality of side-facing cameras and/or wide-angle cameras on a vertical, raised side, or overhang of the platform. The system can further include a sensor in or on the platform and configured to detect that the user has stepped upon the platform. The imaging device can be configured to automatically image a foot of a user after the sensor has detected that the user has stepped upon the platform. A base of the platform can be less than 5 cm in height, less than 4 cm in height, or less than 3 cm in height. The platform can further include a scale configured to weigh the user. The system can further include a communication module configured to communicate with the user about a position of the user's foot on the platform and/or a stage of an imaging cycle. The imaging device can be configured to produce images of the foot within less than 10 seconds, within less than 5 seconds, within less than 3 seconds, or within less than 1 second of the sensor detecting that the user has stepped on the platform. The system can further include a collimator filter configured to achieve a tailored imaging depth. The large area imaging sensor can include a tailored imaging depth such that areas within 75 mm, within 50 mm, or within 40 mm are in focus and areas further away are not in focus. The processor can be configured to automatically detect an ulcer on the user's foot. The large area imaging sensor can include an array of photodetectors. The system can further include a sensor configured to detect a presence of the foot of the user. The imaging device can be configured to automatically begin imaging based upon a detection of the presence of the foot. The sensor can include a load sensor, pressure sensor, a capacitive proximity sensor, a heat sensor, or a light sensor. The system can further include a plurality of load sensors. The processor can be further configured to detect the foot abnormality based upon a force distribution of the foot detected by the plurality of load sensors. The processor can be wirelessly connected to the imaging device.

In general, in one embodiment, a system for detecting a foot abnormality includes a platform configured for engagement with a foot of a user, an imaging device within the platform configured to image the foot of the user when the foot is engaged with the platform so as to gather a plurality of images over time, and a processor connected to the imaging device. The processor can be configured to provide an indication of a changing condition of the foot over time based upon the plurality of images. The system can further include a large area imaging sensor configured to image the foot of the user while the foot is engaged with the platform. The processor can be in the platform. The processor can be remote from the platform. The processor can be configured to issue an alert flag indicating suspicion of a foot abnormality based on the plurality of images gathered by the imaging device. The system can further include a plurality of side-facing cameras and/or wide-angle cameras on a vertical, raised side, or overhang of the platform. The system can further include a sensor in or on the platform and configured to detect that the user has stepped upon the platform. The imaging device can be configured to automatically image a foot of a user after the sensor has detected that the user has stepped upon the platform. A base of the platform can be less than 5 cm in height, less than 4 cm in height, or less than 3 cm in height. The platform can further include a scale configured to weigh the user. The system can further include a communication module configured to communicate with the user about a position of the user's foot on the platform and/or a stage of an imaging cycle. The imaging device can be configured to produce images of the foot within less than 10 seconds, within less than 5 seconds, within less than 3 seconds, or within less than 1 second of the sensor detecting that the user has stepped on the platform. The system can further include a collimator filter configured to achieve an imaging depth. The large area imaging sensor can include a tailored imaging depth, such that areas within 75 mm, within 50 mm, or within 40 mm are in focus and areas further away are not in focus. The processor can be configured to automatically detect an ulcer on the user's foot. The large area imaging sensor can include an array of photodetectors. The system can further include a sensor configured to detect a presence of the foot of the user. The imaging device can be configured to automatically begin imaging based upon a detection of the presence of the foot. The one or more sensors can include a load sensor, pressure sensor, a capacitive proximity sensor, a heat sensor, or a light sensor. The system can further include a plurality of load sensors. The processor can be further configured to detect the foot abnormality based upon a force distribution of the foot detected by the plurality of load sensors. The processor can be wirelessly connected to the imaging device.

In general, in one embodiment, a method of detecting a foot abnormality includes automatically detecting that a foot of a user has engaged with an imaging platform, after the step of automatically detecting, imaging the foot of the user with an imaging device in the imaging platform to produce a plurality of images, and detecting a foot abnormality based upon the plurality of images.

In general, in one embodiment, a method of imaging a foot includes automatically detecting that a foot of a user has engaged with an imaging platform, after the step of automatically detecting, imaging a foot of the user with an imaging device in the imaging platform to produce at least one image, and automatically determining if a foot abnormality is present based upon the plurality of images.

This and other embodiments can include one or more of the following features. The step of automatically detecting can include automatically detecting before a user steps into or after a user steps out of a shower and/or while the user is standing or stepping in front of a sink. The step of imaging can include producing the plurality of images within 10 seconds of when the user has stepped onto the imaging platform. The imaging platform can be positioned in a bathroom. The method can further include notifying the user to reposition the user's foot or notifying the user of a status of an imaging cycle. The method can further include determining a weight of the user with the imaging platform. The method can further include sending an alert flag to a member of a care team at a remote location indicating that a foot abnormality was detected. The step of imaging can include imaging with a large area imaging sensor. The step of imaging can include imaging with a tailored imaging depth of within 75 mm, within 50 mm, or within 40 mm. Imaging can include imaging the plantar surface of the foot. The imaging platform can further include a plurality of wide-angle cameras, and imaging can further include generating a plurality of images of a side or tops of a toe or a side of a heel of the user. The method can further include generating a 3D visual model of the foot of the user based upon the plurality of images. The method can further include displaying an image of the foot abnormality on a remote display. The method can further include displaying a series of images taken over time of the foot of the user on a remote display. A first image of the series of images includes an image of the foot having the foot abnormality and a second image of the series of images includes an image of the foot not having the foot abnormality.

In general, in one embodiment, a device for detecting a foot abnormality includes a platform configured for engagement with a foot of a user, an imaging device within the platform configured to image the foot of the user when the foot is engaged with the platform, a processor connected to the imaging device. The processor is configured to detect a foot abnormality by gathering a plurality of images of a plantar surface and a lateral, medial, or dorsal surface of the foot with the imaging device, stitching the plurality of images together to form a three-dimensional model of the foot, and detecting an abnormality in the three-dimensional model indicative of a foot abnormality.

In general, in one embodiment, a device for detecting a foot abnormality includes a platform having a base configured for engagement with a foot of a user and an edge extending vertically upwards from the base, an imaging device within the base and the edge configured to image a plantar surface of a foot of the user from the base and to image a lateral, medial, or dorsal surface of the foot from the edge, and a processor connected to the imaging device configured to detect a foot abnormality from the captured images.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a schematic showing use of an exemplary foot complication detection system.

FIG. 2 is a schematic showing an exemplary foot complication detection system.

FIG. 3 shows a foot complication detection system for use near a bathtub.

FIG. 4 shows a platform of a foot complication detection system with a raised edge for use near a bathtub.

FIG. 5 shows a schematic of a foot complication detection system for use near a bathtub. The platform has an overhang for imaging the top of the foot.

FIG. 6 shows a schematic of a platform of a foot complication detection system for use near a bathtub. The platform has three raised edges for imaging the front and sides of a foot.

FIG. 7 shows a schematic of a platform of a foot complication detection system with foot shaped cut-outs or contours for receiving the front of a foot.

FIG. 8 shows a schematic of a platform of a foot complication detection system with holes or cavities shaped and sized for guiding and receiving a patient's feet into a desired position.

FIG. 9 shows a schematic of a platform of a foot complication detection system configured to sit in front of a toilet.

FIG. 10 shows a flat mat platform configured to be placed in a bathtub or shower.

FIG. 11 shows a stool platform of a foot complication detection system in front of a toilet.

FIG. 12 shows a block element of a foot complication detection system with sensors and imaging devices next to a bathtub. The block element can image a patient's feet without the patient stepping on the block element.

FIG. 13 shows a block element with sensors and imaging devices next to a sink.

FIG. 14 shows block elements with sensors and imaging devices placed at the corners of a bathtub.

FIG. 15 shows a block element with sensors and imaging devices placed on the side of a bathroom door.

FIG. 16 shows a block element with sensors and imaging devices shaped and sized to partially wrap around the base of a toilet.

FIGS. 17A-17C show exemplary large area imaging sensors. FIG. 17A shows a large area imaging sensor with an array of photodetectors and a lighting element below the array. FIG. 17B shows a large area imaging sensor with an array of photodetectors with a lighting element above the array. FIG. 17C shows a large area imaging sensor with an array of photodetectors with a lighting element within the array.

FIG. 18 is a schematic showing production of a 3D visual model of a foot from a plurality of 2D images.

FIGS. 19A-19B are schematics showing different types of image generation. FIG. 19A shows a schematic of a plantar image of a patient's foot with a foot ulcer. FIG. 19B shows a schematic of a side image of the patient's foot. The side image in FIG. 19B can incorporate data from the image taken in FIG. 19A.

FIG. 20 is a schematic showing production of a 3D visual model of a foot using a 3D model of a standard foot as a basis.

FIG. 21A shows a schematic of a platform of a foot complication detection system configured to perform multiple functions, including a scale for measuring a patient's weight as well as a foot and leg imager.

FIG. 21B shows a schematic of a platform of a foot complication detection system configured to perform multiple functions, including a scale for measuring a patient's weight, a foot and leg imager, and a bathroom mat.

FIG. 22 is a schematic illustration of an automatic foot complication detection system with remote image processing.

FIG. 23 is a schematic illustration of part of an automatic foot complication detection system comparing a series of images of a patient's feet over time. The series shows progression of a potential foot abnormality over time.

FIG. 24 is a schematic illustration of an exploded view of a foot complication detection platform.

FIGS. 25A-25B are schematic illustrations of an exploded view of a foot complication detection platform with a plurality of side-facing cameras. FIG. 25A illustrates the side-facing cameras in a vertical or raised side of the platform. FIG. 25B illustrates the overlapping angle of views of the side-facing cameras for generating stereo images and a 3D model of a user's feet.

### DETAILED DESCRIPTION

Described herein are systems, devices, and methods for detecting early stage foot abnormalities (also referred to herein as foot complications or complications (e.g., complications caused by repetitive stress/pressure, trauma, vascular irregularities, and/or infections, such as an ulcer, callus, fungus, deformed toenail, wound, and/or laceration) to any part of the leg or foot (e.g., the plantar, lateral, medial, or dorsal parts of the foot, toes, toenails, heel, and/or ankle). The system can use images, including images generated within the visual spectrum of light and images generated within a spectrum of light outside of the visual range (e.g., within the infrared spectrum), to identify foot complications. In some embodiments, the system can include a platform that includes a flat mat configured to image the plantar surface of the feet and/or additional element(s) configured to image the lateral, medial, and dorsal parts of feet. In some embodiments, plantar pressure or force distributions and/or temperature/infrared readings can be used in combination with the generated images to detect complications. In some embodiments, the system can be connected via a network for detection of complications and/or can trigger a notification when complications are identified.

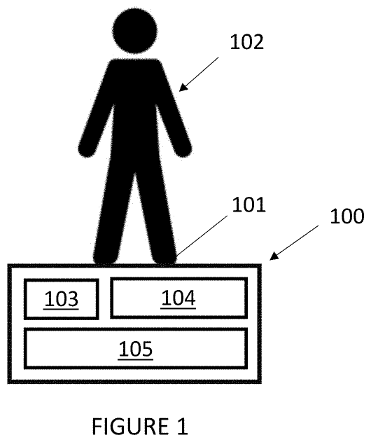

Referring to FIG. 1, an exemplary foot complication system is shown. FIG. 1 shows a detection platform 100 (e.g., a mat or raised surface) configured to screen the bottom of a patient's foot 101 when the patient 102 steps barefoot onto the platform 100 for early indicators and risk factors for foot complications. The platform 100 can include one or more presence sensors 103 to detect the presence of the patient 102 and an imaging device 104 to take an image of the foot 101. In some embodiments, the presence sensors can be one or more load or pressure sensors to detect when a force or pressure is applied (e.g., by the foot) on the platform 100. In some embodiments, the presence sensors 103 can be one or more ambient light sensors to detect when a light in the room (e.g., bathroom) is turned on and/or a shadow is cast over the platform 100. In other embodiments, the presence sensors 103 can be one or more capacitive or other proximity sensors to detect when a patient is close to the platform 100.

The imaging device 104 can be configured to take images of the foot 101 (e.g., of the plantar, anterior, posterior, lateral, medial, and/or dorsal surfaces). The platform 100 can further include a platform processor 105 configured to analyze the images taken with the imaging device 104 to detect foot complications. The one or more presence sensors 103 can be used to detect when a person steps on the platform 100. In some embodiments, this detection can be used to trigger the imaging device 104 and/or platform processor 105. The platform 100 can further include a battery and/or power cord and/or can be configured for wireless charging.

The platform 100 can be used, for example, as a bathmat. To function as a bathmat, for example, the platform 100 can be waterproof and/or water wicking, can include texturing, can include an active drying mechanism, can have a pattern thereon with multiple materials to absorb, or can include light-transmissive sections or light guides within a water-absorptive material. Further, the platform 100 (or the base of the platform, excluding a vertical or raised side, overhang, etc.) can be 5 cm or less in height, such as 4 cm or less in height, 3 cm or less in height, or 2 cm or less in height

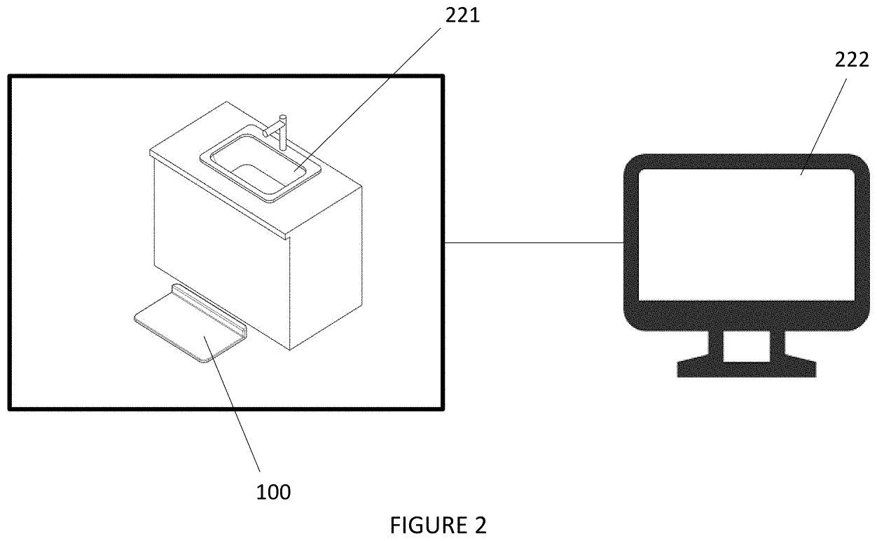

Referring to FIG. 2, in some embodiments, the platform 100 (which can be, for example, in a bathroom next to a sink 221) can be connected to a remote processor 222 (for example, via a connector such as an Ethernet cable connection, wireless internet card, direct internet connection, a cellular connector, Wi-Fi, or Bluetooth). The remote processor 222 can be used in lieu of or in addition to the platform processor 105 in the platform 100 or any other platform described herein. In some embodiments, the platform 100 can be connected to a local or platform processor 105 via a connector, such as a data cable. In some embodiments, for example, the platform processor 105 can combine data from multiple sensors 103 together into one packet (e.g., images from multiple image sensors and/or data from presence sensors), adding additional size and position information based on which sensor(s) 103 the data comes from, while the remote processor 222 can create the visual model and perform the analysis to detect foot complications. In some variations, a system for detecting a foot complication may have multiple processors, such as one or more than one remote processor and one or more than one platform processor. In some variations, platform processor 105 and remote processor 222 may be configured to perform the same or similar functions (e.g., platform processor 105 and remote processor 222 may be redundant and be configured to perform redundant functions). A user may choose which type of processor(s) to use with a system. As used herein, unless otherwise indicated, processor may refer to a remote processor and/or a platform processor.

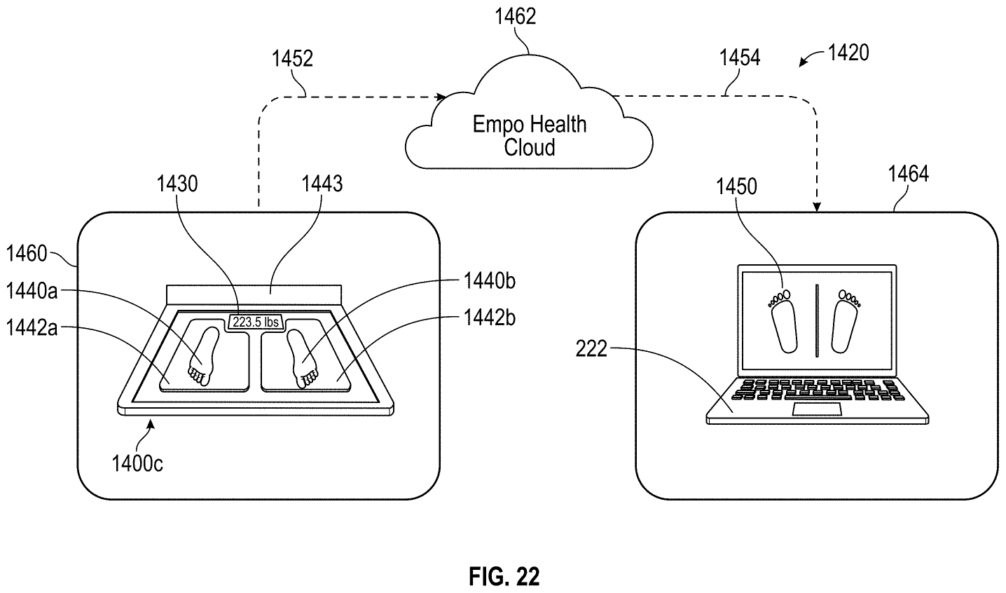

Referring to FIG. 22, system 1420 for detecting a foot complication is configured to issue an alert and/or communicate an alert flag to a patient or a member of a care team at a remote location. The alert flag can be issued and/or communicated to indicate data generation and/or detection of a foot abnormality (e.g., a foot complication). As illustrated in FIG. 22, platform 1400c can take one or more images of a patient's foot (not shown) and/or generate other data at site of use 1460. The platform 1400c can then, via one or more connectors such as a data cable, an Ethernet cable connector or a wireless card, send the one or more images to a processor, send (arrow 1452) the one or more images of the patient's foot and other data taken at platform 1400c at site of use 1460 to internet cloud 1462 (e.g., a first remote processor). Cloud 1462 can store and/or analyze the images and associated data and send (arrow 1454) an alert flag to remote location 1464, such as to remote processor 222 (a second remote processor in this example) or to another remote receiver. Remote processor 222 or another remote receiver may be monitored by a member of a care team, such as a doctor, a nurse, other caregiver, or a family member. Remote processor 222 may generate visual model 1450 showing a visual model of the patient's foot, and a member of the care team may view the visual model 1450. The visual model 1450 may be especially useful for a member of the care team to help determine the nature of a foot complication or foot concern and next steps (if any are needed) to help the patient. In some variations, platform processor 105 or cloud 1462 may generate a visual model, and remote processor 222 may receive the generated visual model, e.g., from platform processor 105 or cloud 1462. In some examples, the alert flag may be sent to remote processor 222 only if a foot complication, foot abnormality, or other concern is detected by system 1420. In some variations, the alert flag can be sent even if a foot complication, foot abnormality, or other concern is not detected, such as whenever an analysis is performed or on a regular basis. In some variations, when image analysis and/or data analysis are performed locally by platform processor 105, platform processor 105 can send an alert flag to cloud 1462 (which can send an alert flag to remote processor 222) or can send an alert flag directly to remote processor 222 (such as if a system for detecting a foot complication is not connected to a cloud). The remote processor can be, for example a computer, a monitor, or a smart phone. The remote processor can be monitored by a member of a care team, such as a doctor, a nurse, or a family member. The alert flag can be, for example, an audible alert (e.g., an alarm, a beep, a phone call, a voicemail) and/or a visual alert (e.g., an email, a colored light, a message, a pop-up, a text.)

Further, the platform processor 105 or remote processor 222 can be configured to send and/or make available raw data, processed or analyzed data, and/or notifications to patients and/or their providers and/or other members of their care team, for example their family. In some embodiments, for example, gathered and/or analyzed data can be accessed through a web browser or application-based service. In some embodiments, the user and/or provider can receive notifications on an app or via text message. In some embodiments, the user and/or provider can receive notifications via a communications module (a local (platform) communication module or a remote communication module), such as a speaker or lights on the platform 100 and/or on the remote processor 222 or other remote receiver. In some embodiments, the notifications can include alerts to the user to reposition the feet for better reading and/or where to reposition the feet to, alerts to indicate the timing in an imaging cycle (e.g., whether the user can move his or her feet/leave the platform), alerts to see a doctor, and/or alerts that a complication has or has not been detected.

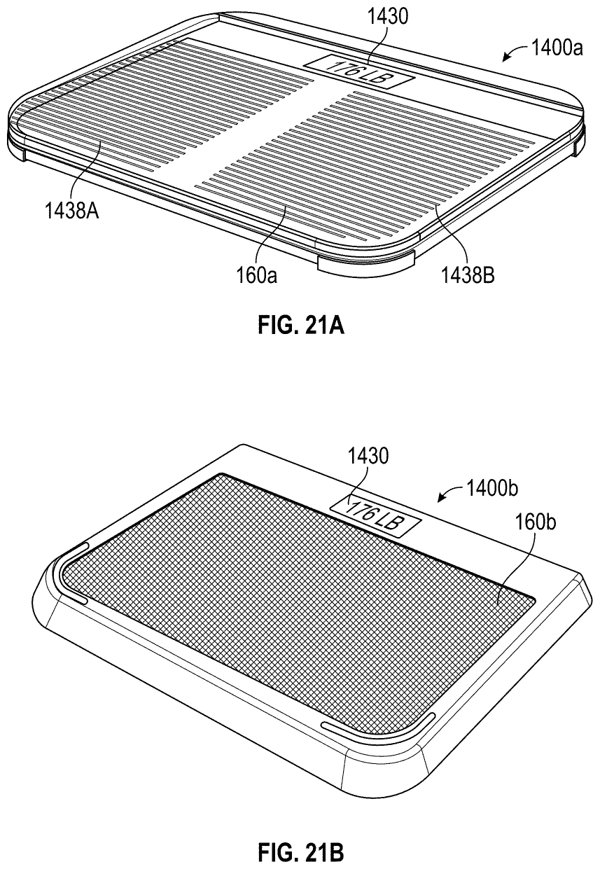

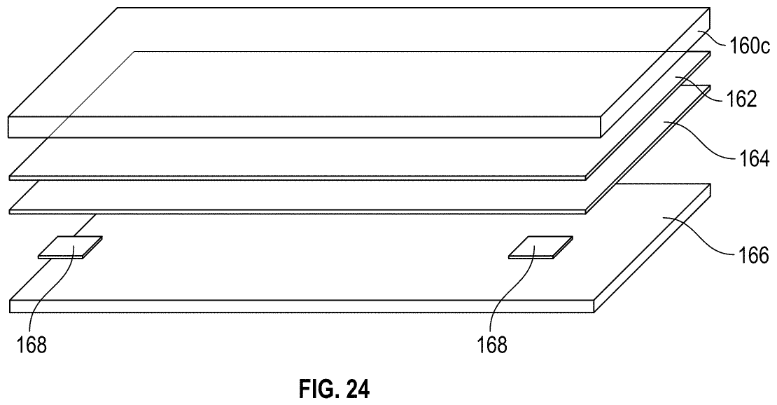

Referring to FIG. 24, in some embodiments, an imaging device (or any other imaging device or system described herein) can include a large area imaging sensor 162, e.g., an imaging sensor that is configured as a two-dimensional array of photodetectors where the size of the sensor is the same as the size of the field of view. The large area imaging sensor can be positioned (e.g., immediately) below the horizontal surface 160c of the platform 100 on which the user stands. The large area imaging sensor can be positioned above support 164 of the platform 100. The imaging device in FIG. 24 also includes one or more than one (2, 3, 4, 5, 6, 7, 8) force transducers or load cells 168 that may rest upon support 164. This and other imaging devices described herein may contain one or more than one large area imaging sensor with these and other features described herein (e.g., each imaging device can be configured as a two-dimensional array of photodetectors where the size of the sensor is the same as the size of the field of view; positioned (e.g., immediately) below the horizontal surface, etc.) Surface 160c on platform 100 may include a protective, non-slip surface, such as made from a polyvinyl chloride (PVC) or a thermoplastic rubber (TPR) material. Surface 160c may be textured, such as with bulges, dots, indents, lines, or waves that prevent a patient from slipping and falling. For example, FIG. 21A shows platform 1400a with surface 160a with textured lines, and FIG. 21B shows platform 1400b with surface 160b with a checkered surface. Any of the surfaces (e.g., surface 160a, 160b, and/or 160c as well as associated structures including image sensors and support materials) can be a continuous surface or discontinuous surfaces. In some examples, a discontinuous surface may have two separate surface regions and act as a foot guide for a patient's feet. For example, FIG. 21A shows separated surface 1438a and surface 1438b configured to separately act as foot guides for placement of a patient's left and right feet. FIG. 22 shows a first large area image sensor 1442a and second large area image sensor 1442b. The image sensors are located under the regions upon which a patient will step. Thus, in some examples, the sensors can be smaller, easier to manufacture, less expensive, allow a more flexible or foldable mat, etc. Load cells 168 on platform 100 can be configured to convert compression or pressure into an output signal. Load cells 168 may be useful as presence sensors or, when a platform is also used as a scale, for determining a patient's weight.

In some embodiments, the large area imaging sensor may advantageously not require the use of lenses for magnification or minification of the field of view. Further, the large area imaging sensor can advantageously complete imaging in less than 30 seconds, such as less than 10 seconds, such as in less than 5 seconds, such as in 3 seconds or less, such as in 1 second or less, advantageously requiring the user to spend only a short amount of time on the platform 100 while still enabling detection of foot complications.

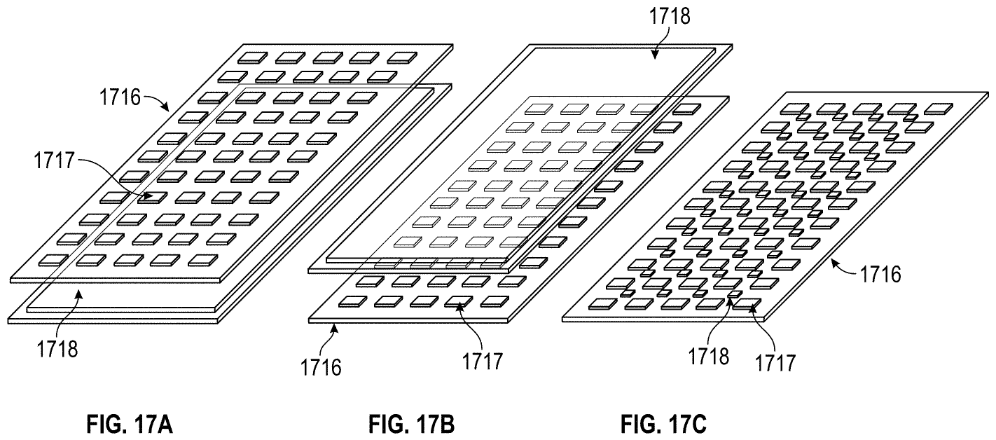

Referring to FIGS. 17A-17C, a large area imaging sensor (e.g., large area imaging sensor 162) can, for example, include an array 1716 of photodetectors 1717 that are positioned over a plurality of lighting elements 1718 (e.g., LEDs or other lighting source) and/or a single lighting element 1718 (e.g., a single backlight (e.g., LCD)). Further, the lighting element 1718 for the platform 100 can advantageously be placed below the array 1716 (as shown in FIG. 17A), above the array 1716 (as shown in FIG. 17B), or within the array 1716 (as shown in FIG. 17C). In some embodiments, the large area imaging sensor can include a filter (e.g., red, green, blue) placed over each photodetector 1717 to ensure a given photodetector 1717 only measures a specific wavelength/color of light. In other embodiments, each photodetector 1717 can be configured to be sensitive to a specific wavelength or color light. Using a filter over each photodetector 1717 or having each photodetector 1717 be sensitive to a specific wavelength can advantageously reduce exposure time. In other embodiments, the lighting element 1718 can be configured to emit a specific wavelength or color of light, which can advantageously reduce the number of photodetectors 1717 required for a given pixel resolution.

In some embodiments, the large area image sensor may be made from one or multiple (e.g., 2, 3, 4, 5, or more) wafer-scale image sensors and the sensors may be butted together or may not be butted together (e.g., they may be separated). In some embodiments, the photodetectors may be discrete components mounted to a printed circuit board. In some embodiments, the large area image sensor may be made, for example, from amorphous silicon deposited onto a substrate (e.g., amorphous silicon deposited onto a substrate and selectively crystalized into a polycrystalline silicon or amorphous silicon deposited onto a substrate and without being selectively crystalized into a polycrystalline silicon), or from other organic semiconductor materials. In some embodiments, the substrate of the large area image sensor can be a thin glass substrate. In this embodiment, a rigid transparent window can be placed above the large area sensor and/or a rigid support can be placed below the large area sensor (e.g., with the large area sensor sandwiched therebetween) to help avoid flexing of the large area image sensor. In other embodiments, the substrate of the large area image sensor can be a flexible (e.g., plastic) substrate, which can advantageously help prevent the large area imaging sensor from breaking even under high user loads.

The large area imaging sensor can include a tailored imaging depth such that areas within 75 mm, such as within 50 mm, such as within 40 mm are in focus and areas further away are not in focus. Imaging within this range can ensure that the entire foot can be in focus in the image while preventing privacy concerns by otherwise focusing on more of the patient's body than necessary. A longer imaging depth could be an issue since the imaging can be performed and/or is designed to be performed (in the bathroom) while a patient is undressed, showering, using the toilet, etc. In some embodiments, the large imaging sensor can include a collimator filter therein or thereover to achieve an imaging depth within the tailored range. The collimator, for example, can be fabricated with carbon nanotubes, with a traditional flat panel manufacturing method, or via micro-machined holes (e.g., with a precision laser cutter). In other embodiments, additional lenses can be used with the large area sensor to achieve an imaging depth within the tailored range. These additional lenses can be, for example, micro lenses, gradient-index lenses, and/or composite lenses made from laminated pieces of materials with different indexes of refraction and placed over the photodetectors of the large area imaging sensor.

Advantageously, the large area image sensor can be less than 20 mm, such as less than 10 mm, such as less than 5 mm, such as less than 3 mm, such as less than 2 mm thick. Additionally, the large area imaging sensor can acquire images quickly (e.g., within 10 seconds, within 10 seconds to 1 second (e.g., within 1 second, within 2 seconds, etc.), within 1 second to 0.1 seconds) of the user stepping on or otherwise engaging with the platform). In some examples, an imaging sensor herein (e.g., large area imaging sensor) can acquire images faster than other imaging modalities can, such as other non-sensing modalities (e.g., contact temperature sending) or a moving scanner imaging sensor. Moreover, the large area imaging sensor can advantageously gather images from a wide range of angles and positions (e.g., rather than requiring the user to stand directly on specific imaging windows).

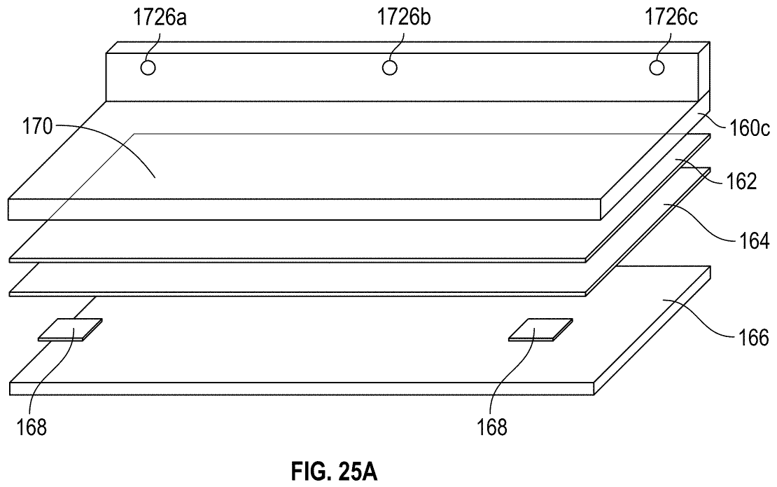

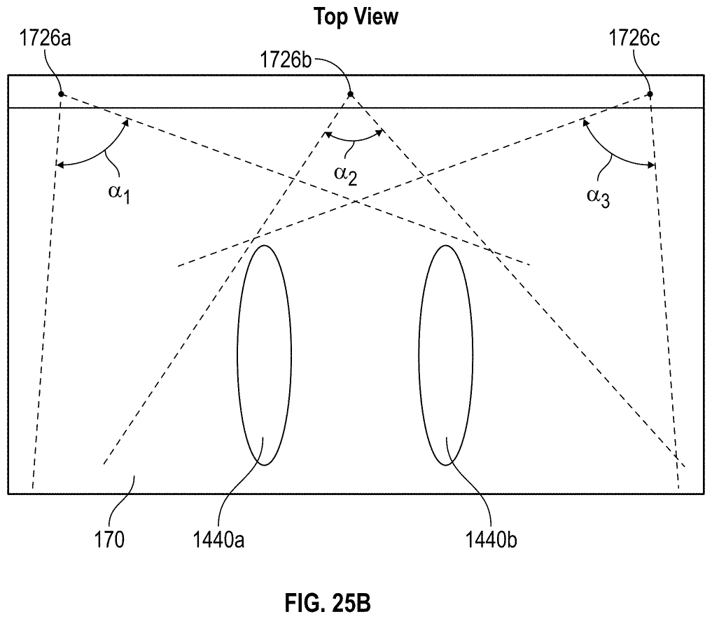

Referring to FIGS. 25A-25B, in some embodiments, the imaging device can include one or more additional cameras positioned on a first vertical or raised side 172 of the imaging device (e.g., above a plantar imaging surface 170). A vertical or raised side may also house electronics for the device. The one or more additional cameras may be in addition to or, in some examples, instead of, the plantar large area imaging sensor 162. FIGS. 25A-25B show, for example, three wide-angle cameras strategically positioned to capture different perspectives on the foot or feet of the patient and may do so simultaneously or sequentially. Other numbers of cameras can also be used and/or placed on other surfaces, such as other side or vertical surfaces. Representative foot placement is shown in first foot location 1440a and second foot location 1440b. (See also FIG. 22). The wide-angle camera lens can capture, for example, from 60° to 180°, such as from 60 to 100, from 100 to 150, from 150 to 170, or from 170 to 180. The wide-angle camera lens can produce a rectilinear image. In some examples, the wide-angle camera lens can be an ultra-wide-angle lens, such as a fisheye lens and may produce a circular rather than a rectilinear image. For example, if the heels are closest to the camera, first camera 1726a captures a region indicated by angle α1, such as the left medial foot from the posterior up to and including the toes, the right lateral foot from the posterior up to and including the toes, the left heel, and the right heel. The second camera 1726b, in turn, captures a region indicated by angle α2, such as the left medial foot from the posterior up to and including the toes, the right medial foot from the posterior up to and including the toes, the left heel, and the right heel. Finally, the third camera 1726c captures a region indicated by angle α3, such as the left lateral foot up from the posterior up to and including the toes, the right medial foot from the posterior up to and including the toes, the left heel, and the right heel. A single camera may image one or more of the plantar aspect of a foot, the heel, the lateral aspect of the foot, ankle, or leg, medial aspect of the foot, ankle, or leg, or any of the toes. Together, however, these cameras can provide stereo images that can be used to generate a 3D model of a user's feet (e.g., by employing measurements made in two or more images taken from different positions).

Non-plantar foot ulcers (typically presenting 5-6 times less frequently than plantar foot ulcers) tend to be concentrated on the toes and heel. In some examples, 3D models create a representation of the toes and/or heels of the patient's feet. The design of the device can keep these areas in view of the stereographic cameras during intended use. In some examples, the cameras (e.g., camera 1726a, camera 1726b, camera 1726c) are in fixed locations on imaging device 104 (and/or relative to one another), and the fixed locations of the cameras is known a priori. Having fixed locations can obviate the first step of many photogrammetric pipelines: registering images to determine real-world positions of the cameras. In some variations, one or more additional cameras may be positioned along a second raised side, a third raised side, or a fourth raised side and/or along a bottom of a top surface of the imaging device (e.g., above the top of the foot). Although described with reference to imaging device 104, any system or imaging device described herein may employ one of more additional cameras positioned on e.g., a vertical or raised side or top side thereof

In some embodiments, the imaging device 104 can include, in addition to or in lieu of the large area imaging sensor, a linear array of photodetectors (e.g., a contact imaging sensor), a plurality of lights, and one or more scanners. The scanner(s) can move the photodetectors along the full length of the foot to produce the image. In other embodiments, the imaging device 104 can include one or more camera sensors with one or more corresponding lenses. In some embodiments, these camera sensors can be manufactured via wafer-level optics processes, which advantageously may allow them to be made more cheaply, more precisely, and in a smaller size.

The imaging device 104 can be designed to fit within a small vertical space, such as 20 cm or less, 10 cm or less, 5 cm or less, 3 cm or less, 2 cm or less, or 1 cm or less.

In some embodiments, the processor (e.g., platform processor 105 or remote processor 222) can build a visual model of the surface of the patient's foot based upon images gathered by the imaging device 104 and can detect one or more irregularities in the visual model.

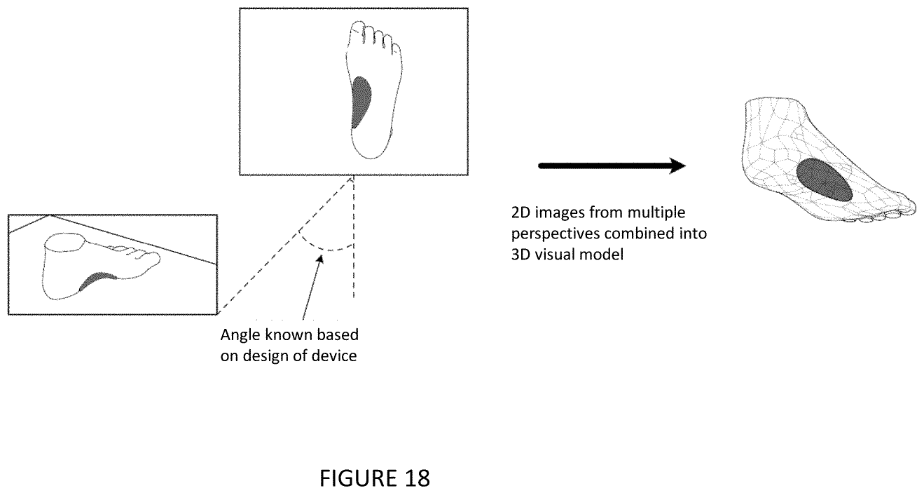

Referring to FIG. 18, in some embodiments, the visual model can be developed by combining all of the images taken by the imaging device 104 to generate a three-dimensional (fully complete or partially complete) visual representation of the surface of the foot, which can then be analyzed for irregularities that may correspond with foot abnormalities or other complications. For example, a visual model can be developed using images from the plantar surface (e.g., with the large area imaging sensor) and from the anterior, posterior, lateral, dorsal, and/or medial surfaces of the foot (e.g., with one or more wide angle cameras). The images from the anterior, posterior, plantar, medial, lateral, and dorsal perspectives, and/or from any other perspectives, taken during one session (e.g., at the same time or slightly spaced apart temporally) can be associated with (or stitched) together. Image identification from the plantar images can allow the orientation and position of the foot to be determined (e.g., can enable identification of the outline of the foot, the location of the foot on the mat, and/or which way the heel and toes are pointing) in order to create a rudimentary foot model located in virtual 3D space. The side images (which can utilize depth information from a previous calibration, stereo information, geometrical perspective with calibration markers on the board, or other range-imaging methods such as time-of-flight and structured/coded light), in turn, can be used to apply further visual information to the relevant surface of the foot model, based on the associated position and orientation from the plantar images.

In some embodiments, as a patient moves around on the mat, images can be taken continuously and/or at regular intervals. Taking images continuously and/or at regular images can enable the visual model of the patient's foot to be incrementally updated. This incremental updating can advantageously produce a higher resolution three-dimensional visual representation of the foot than the sensor resolution would allow for individual images.

In some embodiments a neural network deep-learning-based approach can be used to generate the 3D models. For example, a Volumetric Regression Network can be used and may advantageously not require the use of a 3D Morphable Model. In some embodiments, a semi-global matching algorithm can be used to compute a disparity map for image pairs, providing depth information. This map can then be used to reproject the images onto a 3D point cloud.

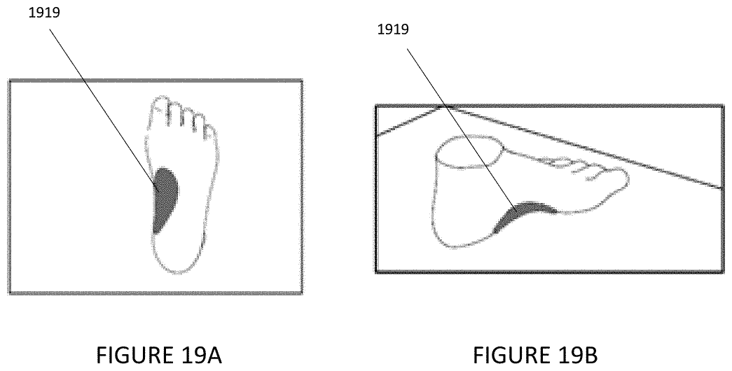

In some embodiments, as shown in FIGS. 19A-19B, the visual model can be developed by tagging the images taken by the imaging device 104 with location and position information of the foot in each of the respective images, allowing a single image view to stand on its own during analysis for foot complications (e.g., enabling analysis with an imaging device that includes only a large area imaging sensor for imaging the plantar surface). That is, by using a plantar image (shown in FIG. 19A), a bare model of the foot can be located and oriented in 3D space. Then, as shown in FIG. 19B, the side image can be mapped directly onto the surface of that model, as the distance from the imaging device 104 to the boundary of the 3D model is known. In the images shown in FIGS. 19A and 19B, there is an ulcer 1919 that spreads from the medial to plantar surfaces because each image is tagged with the position and location information of the foot as the images are taken. In some examples, plantar images can be used without side images and/or without a 3D model to e.g., identify foot structures and foot abnormalities. For example, one or more than one plantar image can be analyzed to identify e.g., toes and heel so that the plantar abnormalities are associated with a location on the plantar surface of the foot.

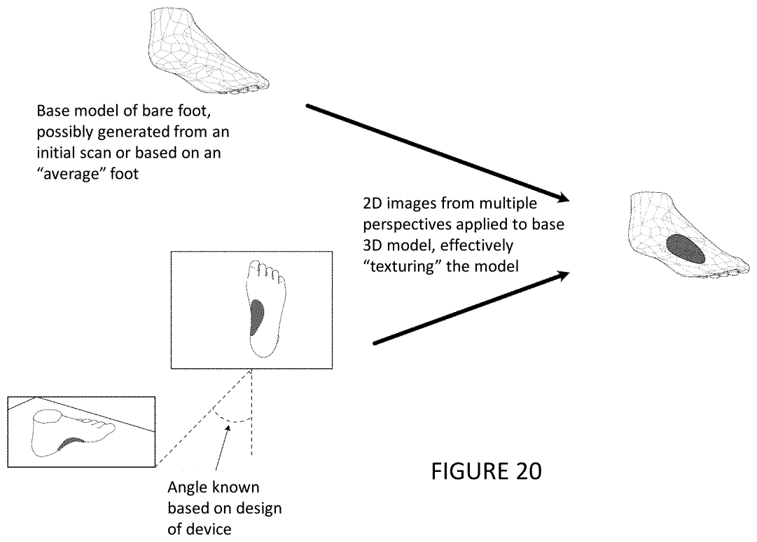

In other embodiments, as shown in FIG. 20, a three-dimensional model of a standard foot can be used as a basis for creating the visual model with the images from the imaging device 104.

In some embodiments, as described above, the visual model can be developed using images from the plantar surface and from the anterior, posterior, medial, dorsal, or lateral surfaces of the foot. In other embodiments, an incomplete visual model can be developed using images from the plantar surface of the foot only.

The irregularities identified by the platform processor 105 or remote processor 222 in the visual model can include, for example, a visual irregularity in a single visual model at a given point in time (e.g., a black spot corresponding to dried blood or necrotic tissue, redness from erythema, a white spot corresponding to a callus, a series of discolored lines indicating fissures from dry skin, or a discoloration under the toenail indicating fungus). The irregularities can include, for example, a difference in the visual model from one point in time compared with another (e.g., the color of a certain spot on a foot changed significantly from week to week, and the discoloration has grown for two days in a row). In some embodiments, the continuous and/or regular images can be used in a time-lapse analysis and/or presentation of the foot (e.g., to determine how a foot complication spread, healed, or otherwise changes over a period of time). Any of the images referred to herein can be black and white images (grayscale) or color images and any of the analyses referred to herein can be performed using black and white images (grayscale) or color images.

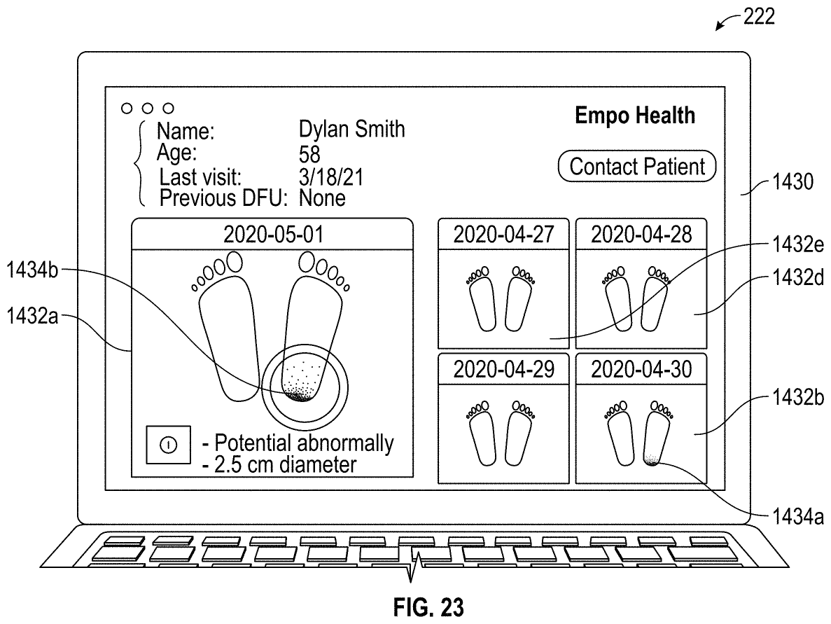

Referring to FIG. 23, remote processor 222 includes display 1430. Display 1430 displays patient information 14 and a series of images 1432a, 1432b, 1432c, 1432d, and 1432e of a patient's feet over time. FIG. 23 shows image 1401a of patient's foot 101 with a 2.5 cm diameter potentially abnormality 1434b. FIG. 23 also shows image 1432b of patient's foot 101 taken just prior to the image 1432a. As shown in FIG. 1432b, the potentially abnormality 1434a has started to develop, but is smaller or less severe than shown in FIG. 1432a. Moreover, the abnormality 1434a/b was not visible in earlier images (1432c, 1432d, and 1432e). By comparing images over time, a care provider can determine various characteristics such as how long a potential abnormality has been on a foot, if the potential abnormality has changed over time, how the potential abnormality has changed over time, how quickly it has changed, if the color of the potential abnormality has changed, etc. Images, such as those illustrated in images 1432a, 1432b, 1432c, 1432d, and 1432e can be automatically generated and analyzed using the systems, devices, and methods described herein. Using the systems, devices, and methods described herein can include the step of displaying a series of images taken over time of the foot of the user on a remote (and/or local) display, wherein a first image of the series of images includes an image of the foot having the foot complication and a second image of the series of images includes an image of the foot not having the foot complication.

One exemplary automated method for analyzing images is through image segmentation/region detection. Clinically relevant information can present in the form of changes in color of a region of the foot and/ or changes in size of those regions. Examples of changes include: a red spot appearing or growing in size across multiple days which may indicate e.g., a region of spreading inflammation; a region of red color shrinking in size may indicate e.g., healing; a region of black color appearing or growing in size may indicate the presence of necrotic tissue and other colors on a region of a patient's foot, such as yellow, could indicate an infection; etc. Provided herein are systems, devices, and methods for taking images across different points in time, automatically annotating the images with regions of interest highlighted, measuring the size of a region of interest, and comparing a size and color from the same region with previous images. These systems, devices, and methods may help care providers and clinicians better understand how different (foot) complications may be progressing.

To detect regions of interest, several processing steps can be used. In one exemplary method of detecting regions of interest, first, images can be color corrected to, for example, account for environment effects (e.g., lighting) on image color or minor manufacturing variations across the different photodetectors in an image sensor. Image sensors can be calibrated against known targets during manufacturing (such as in a factory), and color calibration targets can also be included on the platform (mat) to allow for live color correction in the field during platform or mat use.

Once images have been color corrected, segmentation algorithms, such as thresholding, clustering, and/or neural network based algorithms, can be used to identify regions of the photo image that correspond to feet. Once images have been segmented to identify foot regions, images can be screened to separate out or remove any unusable or partial images.

Next, the size and shape of a foot in an image can be used to identify whether it is a left or right foot and/or whether it belongs to a user in question (as opposed to another user). Users can be filtered out, for example, by weight data from load sensors if included in the mat, but analyzing the images of feet directly can provide a level of redundancy. Once regions in images have been fully segmented and identified, these regions can be aligned with other images in a given capture session, as well as with images from other points in time. This approach can allow images to be analyzed not just alone, but also in comparison with other images.

Finally, foot regions from images can be processed with finely tuned image segmentation algorithms to identify regions of interest on the feet. These regions of interest can then be analyzed for e.g., size, average color, color extremes, color gradient direction, etc., and these measures can be compared with other images from other points in time to understand how the regions of interest are changing. Images can be presented to care providers or clinicians with these regions of interest highlighted and associated with the computed metadata (e.g., additional information about the region of interest, such as a size of an abnormality, length of time the abnormality has been visible, how quickly the abnormality is growing (e.g., how quickly the abnormality is doubling in size), how abnormality color is changing over time, time information when different images were gathered.

In some embodiments, the visual model can be combined with infrared images gathered by the platform to provide additional foot complication detection. For example, near-field infrared can be used to determine blood flow and oxygenation, both of which can be used to identify inflammation or peripheral vascular complications. As another example, mid-field and far-field infrared can indicate temperature in order to identify inflammation (high-temperature) or ischemia (low-temperature). Infrared images can be generated, for example, by reflectance spectroscopy (emitting a light and measuring reflectivity/absorbance from the foot), by emission spectroscopy (measuring photon emissions from the foot), or by fluorescence spectroscopy (emitting a light in order to excite specific molecules/compounds in the foot and measuring the resulting photons released).

In some embodiments, the visual model can be combined with pressure distribution information gathered by the platform (e.g., to include weight in the analysis). The pressure distribution information can, for example, indicate a patient's risk of developing a foot complication over time (e.g., because high pressure points can lead to calluses and ulcers). Thus, for example, high-pressure points in the plantar surface of the foot, particularly ones that increase as time goes on, can be flagged as risks for ulcer development. The information can also, for example, be used to identify a complication (for example, a patient's pressure distribution can change with a wound in the heel, as the body compensates). As another example, the pressure distribution can be used to estimate a patient's posture and loading patterns, tracked over time, to identify key changes that may indicate that a patient's musculoskeletal system is undergoing atrophy due to a progression of neuropathy.

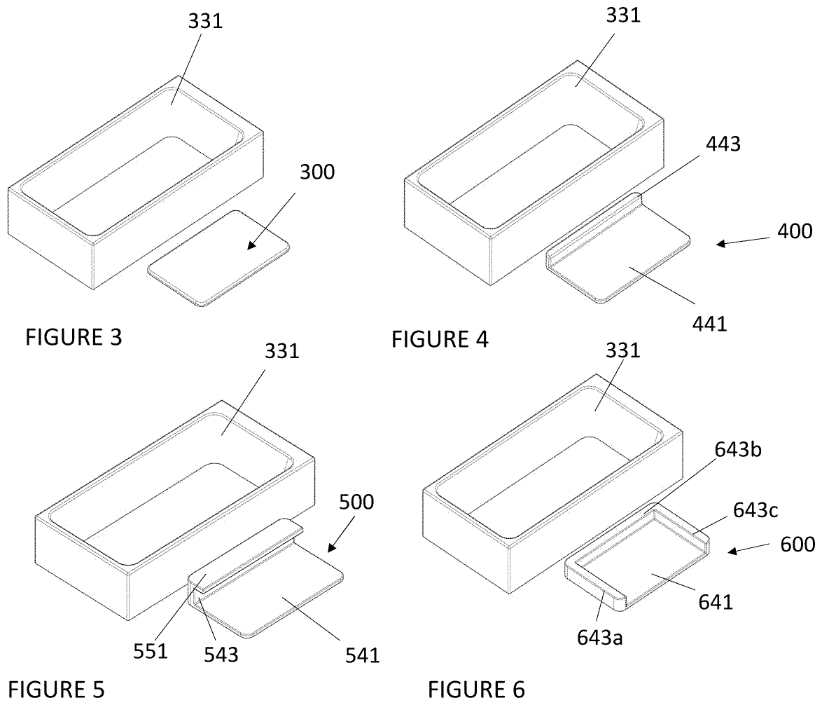

Additional exemplary platforms similar to platform 100 are shown in FIGS. 3-16.

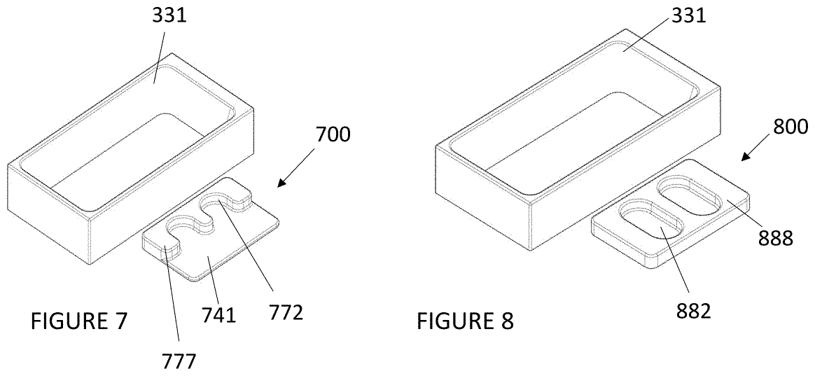

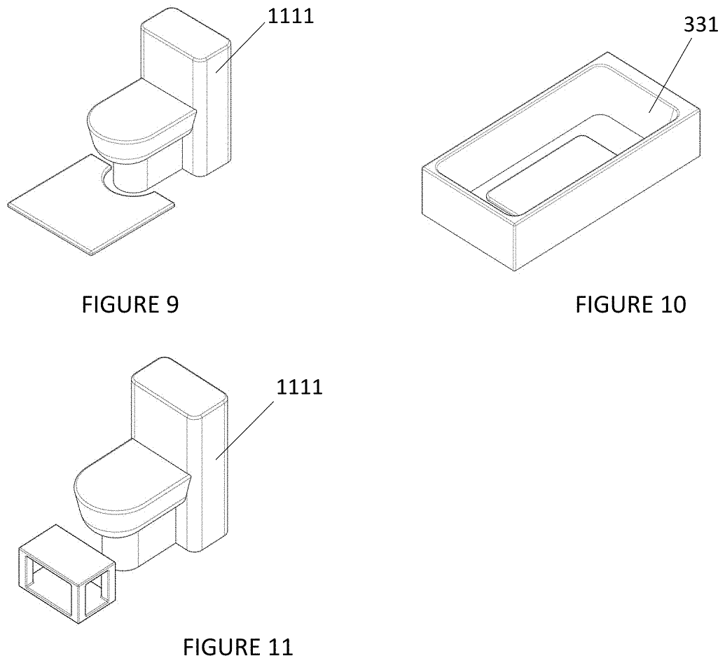

FIGS. 3-8 show platforms positioned adjacent to a shower or bathtub 331 (though each of the platforms could be positioned adjacent to a sink as shown in FIG. 2 or conforming to a toilet base as shown in FIG. 9). As shown in FIG. 3, platform 300 is a flat mat (e.g., a mat having a thickness of less than 50 mm, such as less than 40 mm, such as less than 30 mm) positioned in front of the shower or bathtub 331. As shown in FIG. 4, platform 400 includes a flat mat 441 with a raised edge 443 that is positioned against the bathtub 331 (e.g., so as to avoid tripping thereover). The flat mat 441 can include an imaging device therein configured to image the bottom of the foot while the raised edge 443 can include an imaging device therein configured to image the front, sides, and/or top of the foot. As shown in FIG. 5, platform 500 includes a flat mat 541 with a raised edge 543 having an overhang 551 to better image the top of the foot. As shown in FIG. 6, the platform 600 includes a flat mat 641 with three raised edges 643a,b,c to better image the front and sides of the foot. As shown in FIG. 7, the platform 700 includes a flat mat 741 with a raised element 777 with cut-outs 772 configured to conform to or closely follow the contour of the front of the foot. The raised element 777 can include an imaging device therein configured to image the front, sides, and/or top of the foot. As shown in FIG. 8, the platform 800 includes a flat mat 841 with a raised top layer 888 having holes 882 (also referred to herein as cavities or indents) therein configured to enable the user to stand therein. The holes or cavities extend only partway through the platform or map. The raised top layer 888 can advantageously image all the way around the lateral surfaces of the foot when the user is positioned on the platform 800.

Additional platform designs are shown in FIGS. 21A-21B and FIG. 22. In some variations, any platform as described herein can perform other functions, in addition to performing imaging functions and analysis. For example, platform 1400a in FIG. 21A, platform 1400b in FIG. 21B, and platform 1400c are combined scale and foot complication detectors and include a scale for determining a patient's weight as well as image sensors for detecting a foot complication. The patient's weight may be displayed to the patient on display 1430. A scale may have a piezoelectric transducer that compresses and produces an electric current when a patient steps on the platform 1400c. In some variations, display 1430 may display other information, such as an alert flag that indicates the patient may have a foot complication or should seek medical attention. FIG. 21B is additionally configured as a bathroom mat (bath room mat), such as for use outside of a bathtub, shower, or sink.

Other platform designs are possible. For example, as shown in FIG. 9, the platform 900 can be a flat mat positioned and/or conforming to the base of toilet 1111. As shown in FIG. 10, in some embodiments, the platform 1000 can be a flat mat configured to be placed in a bathtub 331 or shower. As shown in FIG. 11, the platform 1100 can be a stool configured to be placed in front of toilet 1111.

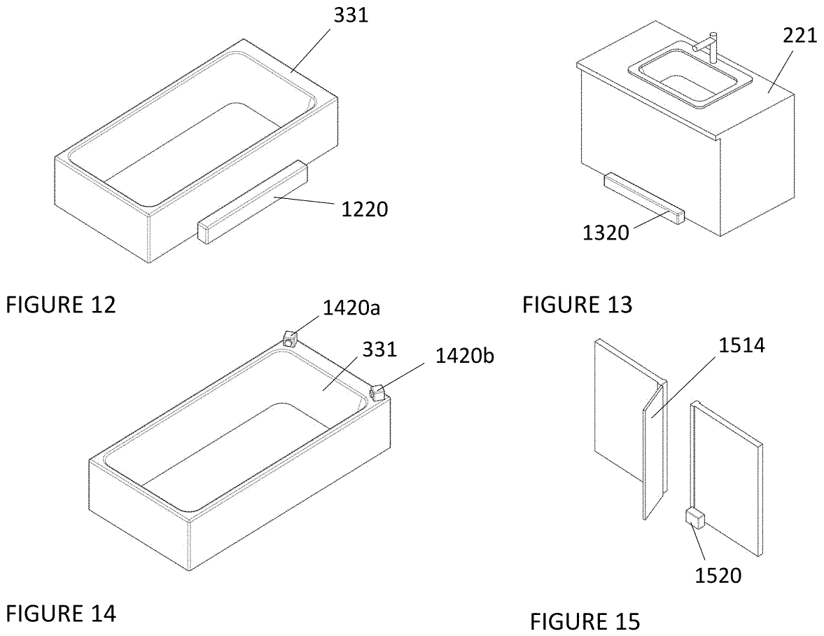

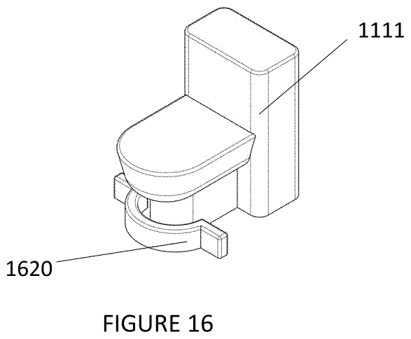

In some embodiments, the platform can be replaced with a block element (including the sensors, imaging device, and/or other features of the platform as described herein) that is configured to be placed in the bathroom, but not stepped upon. For example, as shown in FIG. 12, an elongated block element 1220 can be placed next to the bathtub 331. Similarly, an elongated block element 1320 can be placed next to the sink 221, as shown in FIG. 13. In other embodiments, one or more block elements 1420a,b can be placed at the corners of the bathtub 331, as shown in FIG. 14. One or more block elements 1520 can be placed on the side of the bathroom door 1514 as shown in FIG. 15. One or more block elements 1620 can be placed around the base of the toilet 1111 as shown in FIG. 16.

Advantageously, the systems described herein can enable passive visual monitoring for foot complications. Passive monitoring (i.e., monitoring that does not require activation or input by an individual, such as the patient) can advantageously help ensure patient compliance. Visual monitoring can advantageously automate the current standard of care for foot complication detection and can provide the user (e.g., the medical provider) with detailed medical information regarding the patient's disease state.

Additionally, the systems described herein can advantageously be placed in the bathroom because, while many patients at high risk for ulcers are told to consistently wear shoes, patients tend to still be barefoot in the bathroom, thereby enabling imaging of the feet and monitoring for foot complications.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for detecting a foot abnormality, comprising:
a platform configured for engagement with a foot of a user, wherein a base of the platform is less than 5 cm in height;
an imaging device within the platform, wherein the imaging device comprises a large area imaging sensor configured to image the foot of the user while the foot is engaged with the platform, wherein the imaging device comprises a two-dimensional array of photodetectors, and wherein a size of the large area imaging sensor is about the same as a size of the field of view; and
a connector connected to the imaging device, wherein the connector is configured to communicate with a processor to detect a foot abnormality from at least one image gathered by the imaging device,
wherein the large area imaging sensor includes a tailored imaging depth such that areas within 75 mm are in focus and areas further away are not in focus.

2. The system of claim 1, further comprising the processor.

3. The system of claim 2, wherein the processor is in the platform.

4. The system of claim 2, wherein the processor is remote from the platform.

5. The system of claim 1, wherein the processor is configured to issue an alert flag indicating suspicion of a foot abnormality based on the at least one image gathered by the imaging device.

6. The system of claim 1, further comprising a sensor in or on the platform and configured to detect that the user has stepped upon the platform, wherein the imaging device is configured to automatically image a foot of a user after the sensor has detected that the user has stepped upon the platform.

7. The system of claim 1, wherein the base of the platform is less than 4 cm in height.

8. The system of claim 1, wherein the platform further comprises a scale configured to weigh the user.

9. The system of claim 1, wherein the imaging device is configured to produce images of the foot within less than 10 seconds of the sensor detecting that the user has stepped on the platform.

10. The system of claim 1, further comprising a collimator filter configured to achieve a tailored imaging depth.

11. A system for detecting a foot abnormality, comprising:
a platform configured for engagement with a foot of a user;
an imaging device within the platform, wherein the imaging device comprises a large area imaging sensor configured to image the foot of the user while the foot is engaged with the platform, wherein the imaging device comprises a two-dimensional array of photodetectors, and wherein a size of the large area imaging sensor is about the same as a size of the field of view, wherein the large area imaging sensor includes a tailored imaging depth such that areas within 75 mm are in focus and areas further away are not in focus;
a sensor in or on the platform and configured to detect that the foot of the user has engaged with the platform, wherein the imaging device is configured to automatically image the foot of the user after the sensor has detected that the foot of the user has engaged with the platform; and
a connector connected to the imaging device and the sensor, wherein the connector is configured to communicate with a processor to detect a foot abnormality from at least one image gathered by the imaging device,
wherein the imaging device is configured to automatically begin imaging based upon a detection of the presence of the foot.

12. The system of claim 11, further comprising the processor.

13. The system of claim 11, wherein the processor is in the platform.

14. The system of claim 11, wherein the processor is remote from the platform.

15. The system of claim 11, wherein the processor is configured to issue an alert flag indicating suspicion of a foot abnormality based on the at least one image gathered by the imaging device.

16. The system of claim 11, wherein a base of the platform is less than 4 cm in height.

17. The system of claim 11, wherein the platform further comprises a scale configured to weigh the user.

18. The system of claim 11, wherein the imaging device is configured to produce images of the foot within less than 10 seconds.

19. The system of claim 11, further comprising a collimator filter configured to achieve a tailored imaging depth.

20. A system for detecting a foot abnormality, comprising:
a platform configured for engagement with a foot of a user;
an imaging device within the platform, wherein the imaging device comprises a large area imaging sensor configured to image the foot of the user while the foot is engaged with the platform, wherein a size of the large area imaging sensor is about the same as a size of the field of view, wherein the large area imaging sensor includes a tailored imaging depth such that areas within 75 mm are in focus and areas further away are not in focus;
a sensor in or on the platform and configured to detect that the foot of the user has engaged with the platform, wherein the imaging device is configured to automatically image the foot of the user after the sensor has detected that the foot of the user has engaged with the platform; and
a connector connected to the imaging device, wherein the connector is configured to communicate with a processor to detect a foot abnormality from at least one image gathered by the imaging device,
wherein the imaging device is configured to automatically begin imaging based upon a detection of the presence of the foot.
